(19) United States
(12) Patent Application Publication
(10) Pub. No.: US 2009/0188047 A1
(43) Pub. Date: Jul. 30, 2009

(54) Body support system for a variety of purposes

(76) Inventor: Christopher Brooks Morse, Vancouver (CA)

Correspondence Address: Christopher Morse, 636 West 26th Avenue, Vancouver, BC, V5Z 4H6, CA

(21) Appl. No.: 12/319,684

(22) Filed: Jan. 9, 2009

Related U.S. Application Data
(60) Provisional application No. 61/019,848, filed on Jan. 9, 2008.

(51) Int. Cl. A47C 16/00; A47C 20/08; A47C 27/14; A61M 1/00

(52) U.S. Cl. 5/655.9; 5/657; 604/317

(57) ABSTRACT

The invention is a bodily support system comprised of three dimensional forms designed to place the human body in various positions in order to facilitate a number of physical activities. The forms 1, 2, 3, 4, 5, 6 and 7 all work together as a system as well as individually. Some of the forms fit together and can be used together in pairs or threes. Forms 2 and 3 and forms 6 and 7 are good examples. Others such as form 1 for example, are useful on their own.

### CROSS REFERENCE TO RELATED APPLICATIONS

NONE

### FEDERALLY SPONSORED RESEARCH

NONE

### SEQUENCE LISTING OR PROGRAM

NOT APPLICABLE

### BACKGROUND OF THE INVENTION

1. Field of Invention

This invention relates to ergonomic devices for the purpose of exercise, relaxation, therapy and sex.

2. Prior Art

### Body Support Devices

There is a great number of varying support devices. The problem with most of these devices is that although they do support the body in a more general sense. When it comes to more specific physical activities they fall short. The wedge of foam for instance in U.S. Pat. No. 4,635,306, or the wedges that are commonly seen supporting the back as ones sits in bed as an example, falls short when it is applied to more specific tasks of the body, such as certain physical exertions that are involved in yoga, Pilates, sitting and even certain sexual positions.

The limitation with both the wedge of foam and/or a rectangular block of foam is that they only possess flat planar surfaces while the human back curves as does the thigh. In U.S. Pat. No. 4,905,330, a limitation that we see is that the cushions are shaped with planes on the surfaces of rectangles and wedges. The limitation of this is that it does not conform ergonomically to the body.

The other limitation with U.S. Pat. No. 4,905,330 is that the invention doesn't help the user with the exercise itself. In the Pilates leg work exercises, there are extended periods of time during the exercise session that legs stay in the air. During this period it would be helpful to new users and weaker users to place some sort of support directly under the lower back to help raise the legs. There are wedges of foam that are used for this purpose but they are again limited by the use of flat planes. As a person's back is placed on this plane for exercise, such as leg work in Pilates, a great deal of pressure is placed upon the small of the back as the legs are moved from a floor position to a position where the legs are raised. Further, when the legs do reach this upper limit because of the natural curvature of the spine it becomes more difficult to keep the legs in the upper position as pressure from the flat plane of foam acts as a loaded spring that places pressure upon the lower spine and forces the legs down to the ground.

The leg work in Pilates is very similar to a number of sexual positions. There are however a few differences in regards to sexual acts than that of the other physical movements. It is here that shows another instance of the limitation of the flat plane of foam. As a person approaches the experience of sexual orgasm, the body has a tendency to curl the pelvis upwards and push the small of the back down into underlying support or bedding. A flat plane of foam makes this movement more difficult as the small of the back pushes against the flat foam and in so it takes more force to curl up the pelvis.

The wedges in U.S. Pat. No. 6,925,669 are marketed to perform this very position. Unfortunately, it is the flat plane foam forms that hinder this natural tendency of the body to curl up the pelvis and push the small of the back down into the under support and therefore hinders the performance of this invention to operate to its fullest. For the opposite position where a woman is on her stomach and the wedge is used to prop up her buttocks into a more accessible position for intercourse, the simple straight edge of the wedge digs into her stomach.

We should mention U.S. Pat. No. 6,675,417 B2 as it is a device that is designed out of foam to help contraception. It has attachments that rise up the parts of a woman's torso even higher than a simple block of foam, but once again it falls down due to its planar qualities.

### Support Cushions Staying Together

There are times when it is useful to have two or more cushions within a bodily support system that stack or work together to achieve a goal. A limitation with a number of body support systems is that cushions that are stacked upon one another can slip as the movement of a body upon the cushions can dislodge them out of place. There are a number of ways of dealing with this issue. In U.S. Pat. No. 6,925,669 the inventor dealt with the issue by using a material that when it came into contact with itself created a lot of friction between the surfaces and helped to stop the pillows from being dislodged. The problem with this method is that one must always use the same material in all the versions of the support systems. It is not a particularly good material for the purpose of soaking up sweat or fluids as it can feel a bit sticky to the skin. With nylon materials, it can be challenging to create good colors in comparison to many other textiles. And the end solution is not always met as the activity, if vigorous can dislodge the cushions easily. The cushions also become less able to stay in place as they are washed repeatedly.

The problem has been dealt with also in U.S. Pat. No. 4,171,549. In this invention the stuffing of the support system is of a beaded material as can be seen within beanbags chairs. This allows one cushion to conform to the underlying cushion adding to it some ability to stay in place. They also use different forms that go together in the manner a jigsaw goes together, where each piece is specifically designed to fit into one another. The problem here is that the cushions lose out on their ability to support the body due to the bead stuffing and physical activities become more difficult if not impossible.

In U.S. Pat. No. 4,987,625 the problem is again dealt with by having a number of cushions all connected to each other along a common axis. This does not allow a user to wash just one cushion and the un-stuffing and re-stuffing of the cushion becomes quite a challenge.

### Exercise Apparatus

The classic exercise bench or weight lifting bench has a number of limitations. One of the greatest of these is that the average bench is very heavy, as it has to be made to support a user as well as the weights that the person using the bench is holding. Furthermore it has to be strong enough to hold the user in a number of bench destabilizing exercises such as leg lifts and sit ups, where the weight of the user is distributed differently in the initial phase of the exercise than the later stage of the exercise. A good example of this type of bench would be the U.S. Pat. No. 5,882,283. Most exercise benches are made with an underlying frame made of either wood and/or metal, such as the bench seen in U.S. Pat. No. 7,285,077. This makes them very heavy and extremely expensive to ship. This in turn make it difficult to sell the benches on the Internet where the buyer often pays for the shipping of the product, or it is hard to transport to the buyers home as it can be quite a feat to carry it into the home. The benches are also known for being unsightly and great at making your home look less than classy. There is also the common complaint of users stubbing their toes on the metal footings.

Furthermore, the average weight bench or exercise bench can take up a good deal of space, making it not a good solution for people who live in smaller spaces. There are folding benches, but these are often still too heavy for some people such as women to move even in the folded formation.

Yet another issue with the benches is that the users prefer the backs to incline and decline in that many exercises are not possible on a straight flat bench. This is referenced again with the U.S. Pat. No. 5,882,283, which shows a bench with a number of differing back positions. The addition of the incline adds further weight to the bench, accentuating the limitations as discussed above.

### Incontinence and Physical Movements

Incontinence is an issue of great concern and embarrassment for those inflicted. Physical exercise, sexual expression, sleeping and sitting for a few examples can become difficult and embarrassing. The soiling of seating, beds and equipment, vehicles and cushions can cause great damage to these items as well as place an emotional stress upon those doing the soiling.

Many people place themselves upon an absorbent under pad or use a diaper. Both of these methods can be very obvious in nature. Diapers can leak and have negative connotations due to their use on babies and can often be seen upon a wearer. Absorbent under pads on the other hand are very noticeable as they often sit right out upon the protected surface. Both these methods hold a measure of embarrassment to the users.

The problem with patents such as U.S. Pat. No. 6,727,403 B1 which is an absorbent material, is that for some people with incontinence, they never know when or if an accident will occur. With just the use of materials such as these one must either wear a diaper or pad under their clothing or be placed upon a visible pad constantly. It is possible for some users that the incontinence may be an occasional occurrence where it may seem like over kill to constantly wear a diaper of be placed upon a pad.

Another limitation with the use of the absorbent materials placed closely to the skin is that some people can have allergies to the material within the absorbent pad.

In the instance of the handicapped, the incontinence may be addressed with the insertion of a catheter. The capture of urine escaping the catheter may only be an occasional problem, which the user may not want to address with the constant wearing of a diaper or by being placed upon an absorbent pad.

### Stimulation Devices

Another way of dealing with incontinence is with the use of sexual stimulation. The stimulation of the body to orgasm is known to help increase the muscular development within the urinary system of the body which in turn improves the ability of the unfortunate sufferer to hold their fluids. The use of most vibrators involves a hand held device and the manual stimulation of the genitals to the point of orgasm.

The limitation of most vibrators is that they are not designed to be used without the use of one's hands to hold the vibrator in place. It is further known that exercises such as Kegels, which is the tightening and release of the PC muscles of the lower abdomen as well as exercise in general help the body develop the muscles used to retain fluids. The use of a vibrator during the exercises as well as during sexual intercourse can lessen incontinence. It would be natural for the devices, which help people exercise, and/or have sex, should as well help one use a vibrator or sexual stimulation in such a manner as to enable the user to have no need to use his or her hands. Sadly, there is to our knowledge no such device available.

### OBJECTS AND ADVANTAGES

### Body Support Devices

The invention is helpful as it possesses curves and peaks that are designed to hold the body in a more supportive way than U.S. Pat. No. 4,635,306. It helps to lift limbs, support crunches and sit ups, position the body for sexual activity, relaxation, stimulation, yoga and Pilates.

The invention curves as the human back and thigh, unlike in U.S. Pat. No. 4,905,330 the invention conforms ergonomically to the human body and actually helps the user with exercises or movements.

The invention overcomes another limitation of U.S. Pat. No. 4,905,330 as it helps the user with the exercises itself. As in Pilates leg work exercises, the invention helps the user place and maintain their legs in the air for extended periods of time as the invention places support directly under the lower back. Its curve further facilitates this movement.

In regards to sexual positions such as woman on her back, as in FIG. 6, the invention allows the back to curve as the pelvis curls upwards and helps the angle of penetration, bettering the wedges of foam detailed in U.S. Pat. No. 6,925,669 and in the resultant orgasm. Further in another position where the woman is placed on her stomach as in FIG. 18, the edge if the invention doesn't dig into the stomach in use.

The invention is better than U.S. Pat. No. 6,675,417 B2 as it is simply more comfortable.

### Support Cushions Staying Together

The invention needs no special materials as in U.S. Pat. No. 6,925,669 as the forms fit steadily due to form alone or by supporting the body with only one support apparatus.

The invention doesn't use beads of materials such as in U.S. Pat. No. 4,171,549 therefore there are no choking hazards due to possible holes in the cover and it gives a greater support.

There is no common axis with the invention as in U.S. Pat. No. 4,987,625 so the stuffing and cleaning of the invention is easier.

Another possible answer to the problem of cushions moving apart from each other while in use, lies simply in creating forms that either do not need more than one cushion to function, and/or creating forms that relate to each other depending on the exercise and fit into one another in such a manner as to limit the possibility of movement between them, and/or creating forms that do not move when used in conjunction with each other due to how they are positioned under the body weight of the user.

Creating forms that do not need other forms to be used is obvious as all of the forms are designed to be useful on their own as well as in conjunction with other forms, whether on the floor next to the form in question, as form 5 was designed to be used, for example or not.

Creating forms that fit well together like a jigsaw is another useful strategy, as in the relationship between forms 2 and 3, or forms 5 and 6. Both of these examples were created to make it so that although the materials themselves are not adapted to reduce friction, the forms still do not move away from each other during use.

### Exercise Apparatus

The invention is lighter, stronger, cheaper to ship and more versatile in its positioning than both the U.S. Pat. No. 5,882,283 and the U.S. Pat. No. 7,285,077.

The invention is arguable more attractive than most benches and can be reasonably used as furniture, therefore conserving space. There is also no concern about stubbing your toes on the invention, nor is it a problem for the average person to carry it. It is much lighter for shipping and can hold in excess of 600 lbs.

The benefits to the invention are that it is much lighter than the traditional bench allowing for a much reduced shipping cost, weaker individuals are able to move it easily, and it allows women to put it away if needed.

There is no metal frame or supporting base protruding out of the device and so there is no possibility of stubbing your toe or foot on it. It looks a great deal like furniture and it can be blended into the decor easily as opposed to most gym equipment and although it is the same size as a traditional weight or exercise bench it is comfortable to sit on as any other furniture in the house so that it can fit into smaller dwellings as it can double as a chair.

All the benefits of a reclining bench with its adjustability are met with this new invention.

Most of the exercises that performed on the U.S. Pat. No. 5,882,283 can be performed on the invention plus a good number that cannot.

### Incontinence and Physical Movements

The invention in its present preferred embodiment is completely water resistant so incontinence is not an issue. The use of an absorbent pad under a top cover can always be hidden for any unexpected urine without the use of a diaper, a possible problem with U.S. Pat. No. 6,727,403 B1

The benefit to the user is that the absorbent pad is not visible to bystanders. It is there if one needs it but it does not need to be changed daily such as with a diaper system. The foam underneath remains clean and dry if there is a release of fluids. Another advantage to this pocket system is that the removable pocket can be easily replaced with another while washing the first one.

The absorbent material is kept at a good distance from the user helping those with allergies to the gels used in the absorbent pads.

There is also no need for the use of a catheter for some individuals who find this undesirable.

This method also hinders the runoff of urine onto seats, beds and cushions and the environment in which these are placed such as in the home. This is because many conventional methods use a simple waterproof material that allows the runoff of fluids from the protected surfaces onto surfaces and equipment underneath the cushion.

### Stimulation Devices

The invention can hold a vibrator in place in order to experience its therapeutic effects without the use of hands.

The invention is in the method in which a vibrator or stimulator can be attached or applied to the cushions. Vibration or stimulation can be looked at in two different categories; for sexual stimulation and for muscle relaxation. These are two very different applications, with different levels of intensity of vibration, frequency of the vibration and most importantly the proximity of the vibration to the part of the body that we are looking to stimulate.

For the purposes of the relaxation of the muscles of the back, it has been found that a diffused vibration, meaning that its proximity is a farther distance from the surface of the skin as well as a lower frequency of vibration and a lower intensity of vibration is better. It is better to have the vibrator a farther distance away from the body and have the vibrations transfer through the other materials such as foam, textiles and other possible materials.

For the purposes of sexual stimulation, it has been found that a direct stimulation to the genital area, a higher frequency of vibration and intensity is better. It is better to have the vibrator itself as close to the genitals as possible.

### SUMMARY

The invention is a bodily support system comprised of three dimensional forms designed to place the human body in various positions in order to facilitate a number of physical activities.

### DETAILED DESCRIPTION

### Preferred Embodiments

### Form 1

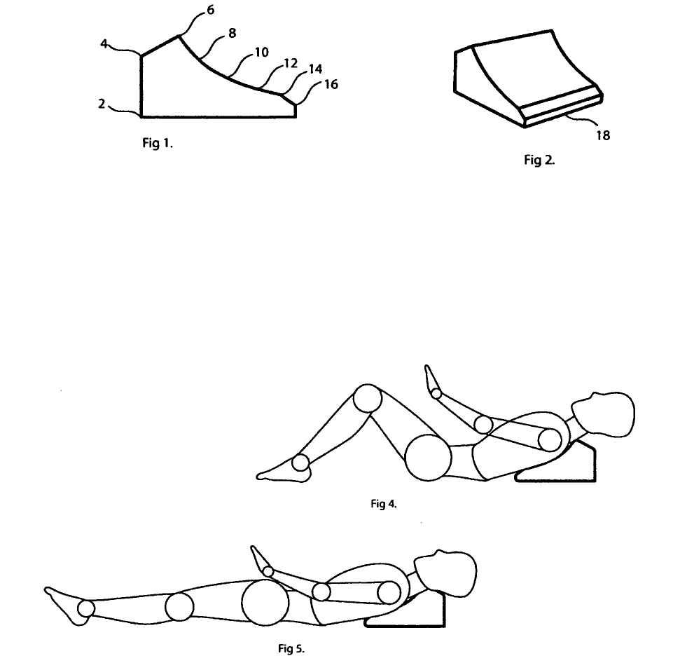

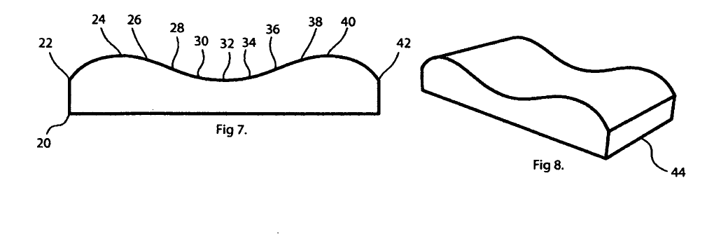

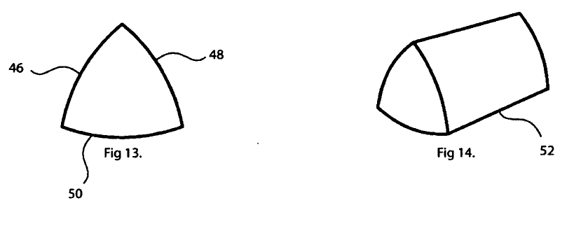

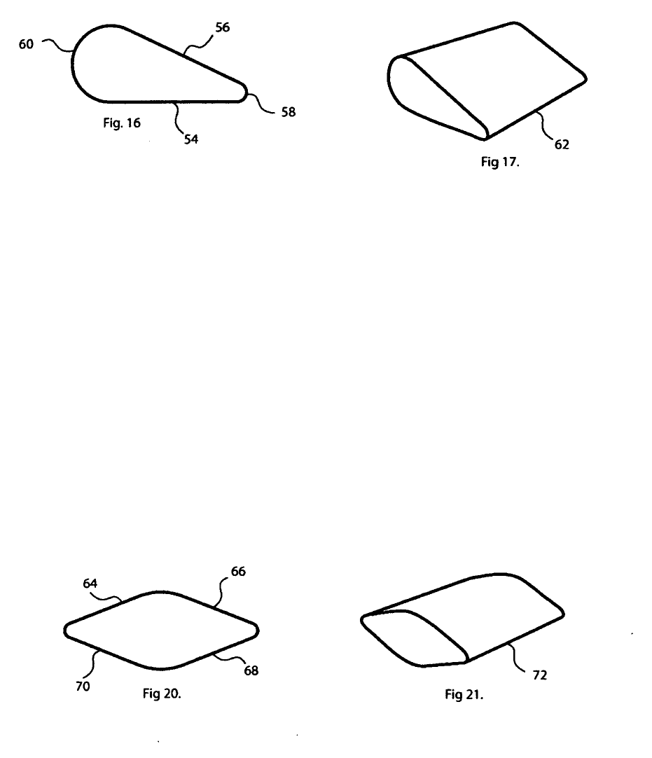

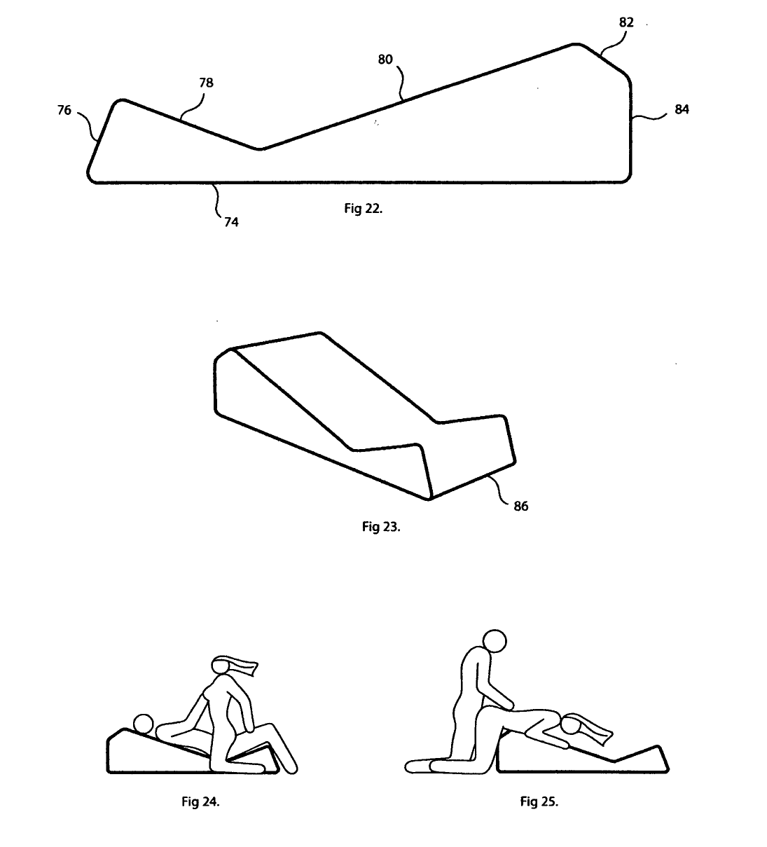

FIG. 2 shows a perspective view of a basic version of form 1. This form is defined by the following points on an X and Y axis, X being on the horizon and Y being on the vertical, starting at the origin point of reference 2 in FIG. 1.

Point 2 is at the origin.

Point 4 is at X=0″, Y=5 and 7/16″
Point 6 is at X=3″ and 5/16″ and Y=7 and ¼″
Point 8 is at X=5″ and 9/16″ and Y=4 and ¾″
Point 10 is at X=7″ and ¾″ and Y=3 and 7/16″
Point 12 is at X=10″ and Y=2 and 9/16th″
Point 14 is at X=12″ and ¼″ and Y=2″
Point 16 is at X=13″ and ½″ and Y=1 and 1/16″

It has been found that although the above coordinates are a definition of the preferred embodiment, each point can vary in its location on the X and Y axis by 1 inch from each other, in any direction on the X and Y axis and still maintain the benefits of the design. The width of the invention is 14″ in the preferred embodiment and it has been found that the invention can vary still function with a width of between 11″ to 30″ as seen in FIG. 2, reference 18.

### Form 2

FIG. 8 shows a perspective view of a basic version of form 2.

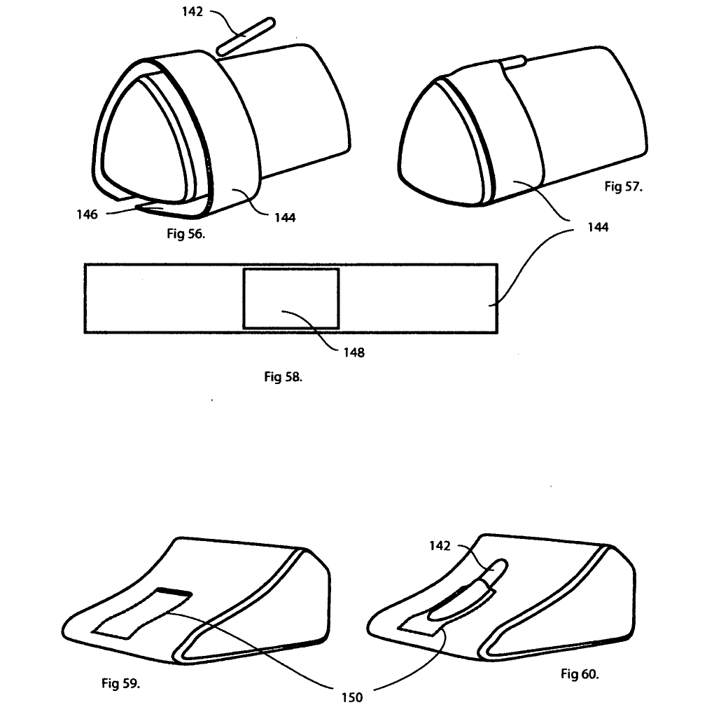

The top surface of the form between the reference numbers 20 and 42 is defined as the following points on the X and Y axis with the origin at reference 20 in FIG. 7.

Point 20 is at the origin.
Point 22 is at X=0, Y=4 and ⅜″
Point 24 is at X=6 and ⅝″ and Y=7 and ⅝″
Point 26 is at X=10 and 1/16″ and Y=7 and 1/16″
Point 28 is at X=13 and 7/16″ and Y=5 and 13/16″
Point 30 is at X=16 and ¾″ and Y=4 and ¾″
Point 32 is at X=20″ and Y=4 and ⅜″
Point 34 is at X=23 and ¼″ and Y=4 and ¾″
Point 36 is at X=26 and 9/16″ and Y=5 and 13/16″
Point 38 is at X=29 and 15/16″ and Y=7 and 1/16″
Point 40 is at X=33 and ⅜″ and Y=7 and ⅝″
Point 42 is at X=40″ and Y=4 and ⅜″

The section of the form between point 24 and point 40 is the section of the invention that promotes orgasm in how it positions the spine and back.

It has been found that although the above coordinates from point 24 to point 40 are a definition of the preferred embodiment; each point can vary in its location on the X and Y axis by 1 inch from each other, in any direction on the X and Y axis and still maintain the benefits of the design. It has also been found that the section between the points 22 to 24 and the section between 40 and 42 can vary more greatly if the points between 24 and 40 are relatively maintained. The width of the invention is 21″ in the preferred embodiment and it has been found that the invention can vary and still function with a width of between 12″ to 30″ as is seen in FIG. 8, reference 44. The origin point 20 is located in the preferred embodiment as being at the base of the invention. This point can vary in its distance to the base to be between 0″ to 30″ and still maintain the invention's orgasmic enhancing capabilities as seen in FIG. 9.

### Form 3

FIG. 14 shows a perspective view of a basic version of form 3.

This Form 3 is defined by the following dimensions below and is referenced in FIG. 13.

Form 3 is a three sided triangular form. Its sides (46, 48 and 50) contain a radius between 14″ and 17″ its preferred embodiment is a side radius of 15 and ⅞″ and its height is between 9″ and 12″ from the lowest point on one of the radius to its highest point at the intersection of the other segments. It is between 12″ and 30″ in length with a preferred embodiment of 20″ (52).

It is very important to know that the radius that is chosen for the sides (46, 48 and 50) are of a similar and corresponding radius to the surface of form 2 as outlined between points 24 and 40 in FIG. 8.

### Form 4

FIG. 17 shows a perspective view of a basic version of form 4.

This form is defined by the following dimensions and referenced in FIG. 16.

A segment (54) of between 9″ and 12″ in length at between 22 to 30 degrees in relation to another segment of between 9″ and 12″ in length (56) that are joined with a radius of between ¾″ and 2″ (58). The segments are also connected at the opposite ends with a radius (60) of between 3″ and 5″. The width of the form is between 19″ and 30″. The preferred embodiment is two equal segments at 11½″ in length that share an angle of 26 degrees with a radius of ⅞″ at one end and on the opposite share a radius of 3 9/16″. The width (62) FIG. 17 of the segment is 21″.

### Form 5

FIG. 23 shows a perspective view of a basic version of form 5.

This form 5 is defined by the following dimensions and referenced in FIG. 22.

A segment (74) is between 35″ to 55″ in length and meets an underlying support such as a floor or bedding and intersects segment (76) which has a length of between 5″ and 12″ with an angle between 60 to 90 degrees rising counter clockwise to the horizon. A segment (78) of between 9″ and 15″ in length intersecting segment (76) at an angle of between 15 and 45 degrees rising clockwise in relation to the horizon. Segment (78) also intersects segment (80) which has a length of between 20″ and 30″ at an angle of between 15 and 35 degrees rising counterclockwise from the horizon. Segment (80) intersects segment (82). Segment (82) has a length of between 0″ and 6″ and can be between 0 and 90 degrees rising clockwise from the horizon. Segment (82) intersects segment (84). Segment (84) has a length of between 4″ and 20″ and in between 60 and 90 degrees rising from the horizon. Segment (84) intersects segment (74).

The preferred embodiment of this form is as follows. A segment (74) is 47 11/16″ in length and meets an underlying support such as a floor or bedding and intersects segment (76) which has a length of 6 7/16″ at an angle of 69 degrees and a radius of 1″. A segment (78) of 12¼″ in length intersecting segment (76) at an angle of 90 degrees with a radius 1″. Segment (78) also intersects segment (80) which has a length of 29 3/16th″ at an angle of 140 degrees with a radius of 2″. Segment (80) intersects segment (82). Segment (82) has a length of 3¾″ and is 125.5 degrees with a radius of 2″. Segment (82) intersects segment (84). Segment (84) has a length of 7 7/16″ and has an angle of 125.5 degrees with a radius of 2″ adjoining segment 82. Segment (84) intersects segment (74).

The width (86) of the form 5 is 20″ in length and is referenced in FIG. 23. There is another preferred embodiment of form 5 which is the same as the above description with a few slight differences as below.

A segment (74) is 49¼″ in length and meets an underlying support such as a floor or bedding and intersects segment (76) which has a length of 10 7/16″ at an angle of 69 degrees with a radius of 1″. Segment (78) of 12¼″ in length intersecting segment (76) at an angle of 90 degrees with a radius of 1″. Segment (78) also intersects segment (80) which has a length of 29 3/16th″ at an angle of 140 degrees with a radius of 2″. Segment (80) intersects segment (82) at an angle of 125.5 degrees with a radius of 2″. Segment (82) has a length of 3¾″. Segment (82) intersects segment (84) at an angle of 125.5 degrees with a radius of 2″. Segment (84) has a length of 11 7/16″. Segment (84) intersects segment (74).

### Form 6

FIG. 27 shows a perspective view of a basic version of Form 6.

Form 6 is defined by the following dimensions and referenced in FIG. 26.

Segment (88) is between 24″ and 38″ in length and intersects segment (90) with an angle of between 15 and 35 degrees. Segment (90) is between 15″ and 26″ in length. Segment (92) is between 9″ and 15″ in length and intersects segment (90). The angle between (90) and (92) is as close as possible to the angle chosen between segments (78) and (80), somewhere in the range between 110 to 160 degrees. Segment (94) is between 0″ and 7″ in length and intersects (92) at an angle between 60 and 110 degrees. Segment (94) intersects segment (88).

The width (96) of the form is between 12″ and 36″ as referenced in FIG. 27.

The preferred embodiment of form 6 is as follows.

Segment (88) is 32⅜″ in length and intersects segment (90) with an angle of 19 degrees and a radius of ¾″. Segment (90) is 22¼″ in length. Segment (92) is 12¼″ in length and intersects segment (90). The angle between (90) and (92) is 140 degrees with a radius of 2″. Segment (94) is 2½″ in length and intersects (92) at an angle of 90 degrees with a radius of 1″. Segment (94) intersects segment (88) with an angle of 111 degrees and a radius of 1″.

The width (96) of the form is 20″ in length as referenced in FIG. 27.

### Exercise Apparatus

### Form 5 and Form 6

FIGS. 23 and 27 show a perspective view of a basic version of forms 5 and 6.

The invention includes the interaction of two forms designed to work together as a unit, forming an adjustable weightlifting/exercise system. By using two forms that are designed to interlock into a number of varying configurations, (FIG. 29) it was found that a great number of the basic exercises could be facilitated.

It should be noted that the preferred embodiment shows a co relation between the Form 5 and the Form 6 where the angle between the segments (78) and (80) on Form 5 is the matching inverse of the angle between the segments (90) and (92) on the Form 6. As referenced in FIGS. 26 and 26, as well as in FIG. 29 reference (102) and (104).

### Alternative Embodiments

### Fluid Collection System

FIG. 38 shows a perspective view of a basic version of the fluid collection system.

Any of the forms could include a removable pocket system as in FIGS. 38, 39 and 40 to collect fluids that are expelled from the body and thereby protects the form from said fluids. A removable, water permeable pocket is attached to a water proof cover which in turn protects the foam or stuffing or form giving material underneath.

The removable water permeable pocket is attached in such a manner that an absorbent pad may be placed within it. The absorbent pad is placed within the removable water permeable pocket and the waterproof cover.

The approximate placement of each absorbent pad is set out in FIGS. 41 (106),42 (108),43 (110),44 (112),45 (114),46 (116)(118),47 (120)(122),48 (124)(126) and 49 (128).

### Method of Stimulation

The invention may also include as a possible embodiment a method in which a vibrator or stimulator can be attached or applied to the forms.

Using the methods below one can place the vibration or stimulating system on the cover. The preferred embodiments for the location of the vibrators or stimulator on the covers is referenced in the FIGS. 50 (130), 51 (132), 52 (134), 53 (136), 54 (138), 55 (140) for each form.

A possible embodiment is to use a strap (144) is made to hold the vibrator or stimulator (142) in place. It contains a non slip material (148) such as rubber or silicone as possible examples and holds the vibrator (142) in place by increasing the friction upon the device by tightening the strap with a fastening device such as a hook and loop fastener (146) as referenced in FIGS. 56, 57 and 58.

A further possible embodiment for the placement of a vibrator or stimulator (142) is to place said vibrator or stimulator within a pocket (150) that is attached to the cover at the locations referenced in FIGS. 59 and 60 using a large buttonhole made in the cover that extends though all the layers, of material to the other side of the cover. On the back side of the cover a pocket is made to hold an average vibrator of varying forms.

A further embodiment for the placement of a vibrator or stimulator (142) is placed within a pocket that is attached to the cover at the locations referenced in FIGS. 59 and 60.

A large buttonhole is made in the cover that extends half way through the layers of material to the interior of the cover. On the inside of the cover a pocket is made to hold an average vibrator of varying forms.

### Operation—Preferred Embodiment

### Form 1

This form is designed for physical exercise, sexual activities with or without a vibrator or stimulating system, reading and comfort, and relaxation through the use of a vibrator or a stimulating system to relax the muscles of the back. It is also designed to assist in orgasm by curving the back during sexual stimulation as in FIG. 6. It is also a supplement to form 6 and 7 as it can help with some additional exercises such as floor work and stretching. The form 1 is designed to correlate to the curves of the body, for this reason the top surface of the form is defined by points that were determined to be of optimum location in reference to the base and in reference to the natural curves inherent in the human body, for the purpose of supporting a number of different locations on the body, most importantly, the lower back, upper back, upper back and neck, top and front of the thigh, and buttocks.

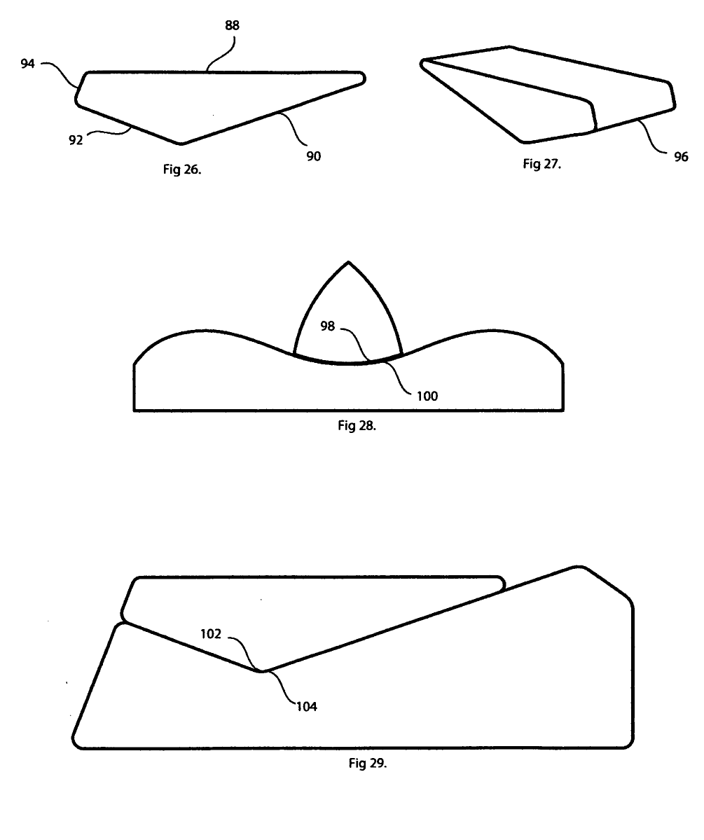

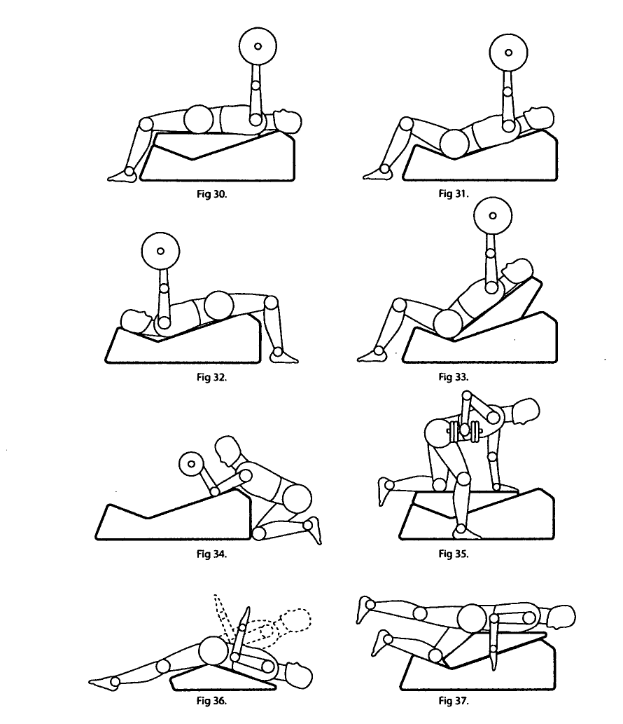

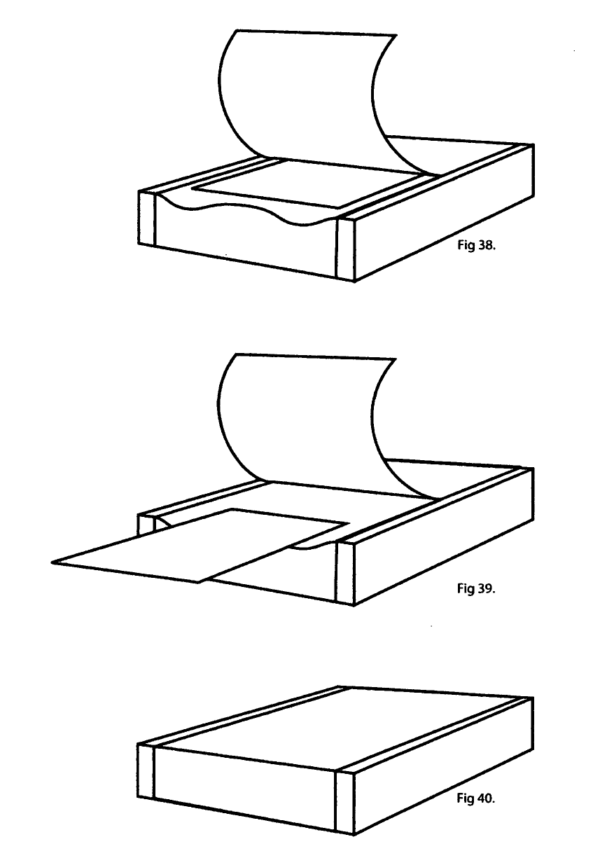

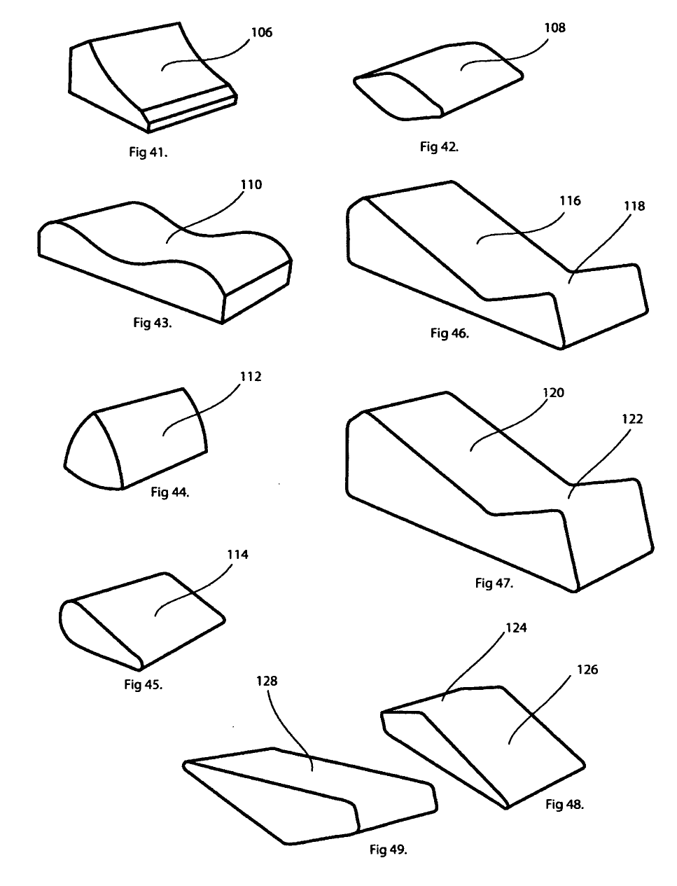

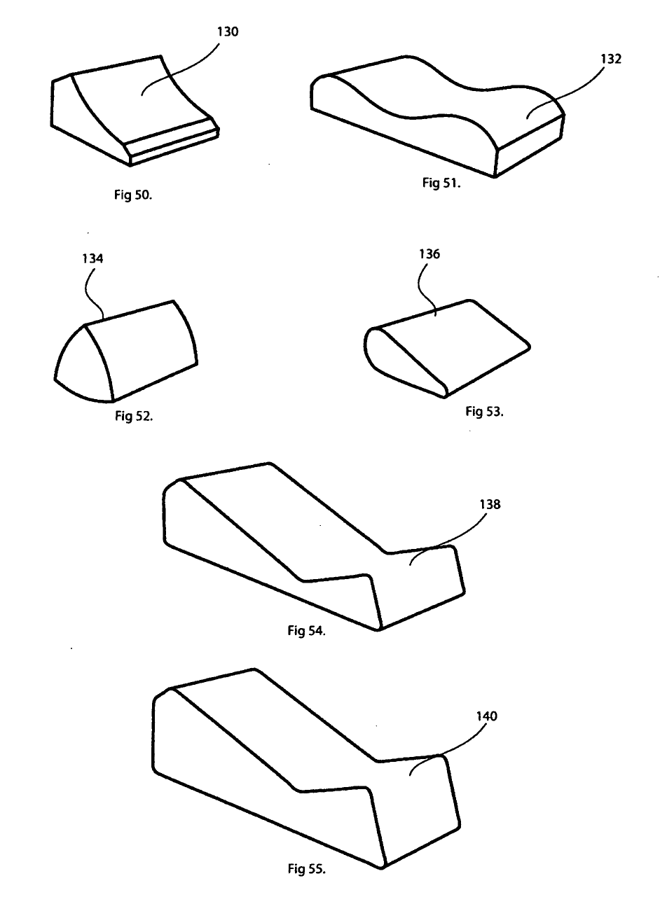

Form 1 is primarily but not exclusively for the following uses:
To be placed under the hips and thighs, while a person is on their stomach as in FIG. 3 for leg lifts, sexual intercourse, sexual stimulation and relaxation through vibration, and other physical activities.
To be placed under the upper back while a person is on their back as in FIG. 4 for sit ups, leg ups and relaxation through vibration as well as other physical activities.
To be placed under the neck and shoulders while a person is on their back as in FIG. 5 for sleeping and reading, yoga and stretching moves and relaxation through vibration as well as other physical activities.
To be placed under the buttocks and lower back while a person is on their back as in FIG. 6 for sleeping, leg lifts, yoga, sexual intercourse, sexual stimulation and relaxation through vibration, and other physical activities.

### Form 2

This form is designed for physical exercise, sexual activities with or without a vibrator or stimulating system, reading and comfort and relaxation through the use of a vibrator or a stimulating system to relax the muscles of the back. It is to assist in orgasm by curving the back during sexual stimulation is designed to work in possible conjunction with Form 3.

It is designed to correlate to the curves of the body, for this reason the top surface of the form is defined by points that were determined to be of optimum location in reference to the natural curves inherent in the human body, for the purpose of supporting a number of different locations on the body. At the point of orgasm the body spasms and curls up the pelvis and curves the back. This form was derived from this position and places the whole back into what a position that we have found naturally promotes orgasm.

Form 2 is primarily but not exclusively for the following uses:
To be placed under the shoulders and spine while a person is on their back as in FIG. 9, for sleeping and reading, yoga and stretching moves and relaxation as it is also excellent for placing the spine in the proper position to enhance and facilitate a sexual orgasm, as well as assist in the act of sexual intercourse as shown in FIG. 10.
To be placed under the hips and thighs, while a person is on their stomach as in FIG. 11 for leg lifts, sexual intercourse, sexual stimulation and relaxation through vibration, and other physical activities.
To place form 3 in the valley between the hills of Form 2 while a person sits astride form 3 in order to be able to cushion the feet and legs as referenced in FIG. 12.

### Form 3

Form 3 is primarily but not exclusively for the following uses:
For a person to ride astride the form 3 as one would a pony for the purpose of sexual gratification with or without the assistance of a vibrator or stimulator which is mounted to the form 3 as referenced in FIG. 15.

Form 3 can be used on its own as well as in conjunction with Form 2, where form 2 becomes an enhancement to the comfort of the user riding Form 3 as form 2 become useful in cushioning the knees as can be seen in FIG. 12.

### Form 4

This form is designed for physical exercise, sexual activities with or without a vibrator or stimulating system, reading and comfort and relaxation through the use of a vibrator or a stimulating system to relax the muscles of the back.

Instead of being the common wedge of foam, the edges that would often be placed under the stomach as in FIG. 18 are rounded out for comfort. This is better for the user as the edges don't push uncomfortably into the body.

Form 4 is primarily but not exclusively for the following uses:
To be placed under the hips and thighs, while a person is on their stomach as in FIG. 18 for leg lifts, sexual intercourse, sexual stimulation and relaxation through vibration, and other physical activities.
To be placed under the buttocks and lower back while a person is on their back as in FIG. 19 for leg lifts, yoga, sexual intercourse, sexual stimulation and relaxation through vibration, and other physical activities.

### Form 5

This form is designed for physical exercise, sexual activities with or without a vibrator or stimulating system, reading and comfort, and relaxation through the use of a vibrator or a stimulating system to relax the muscles of the back. It is also designed to assist in sexual intercourse.

Form 5 is primarily but not exclusively for the following uses:
To be placed under the whole torso while a person is on their back as in FIG. 24 for sleeping and reading, yoga and stretching moves and relaxation through vibration other physical activities including sexual intercourse.
To be placed under the stomach, elbows and hips, while a person is on their stomach as in FIG. 25 for leg lifts, sexual intercourse, sexual stimulation and relaxation through vibration, and other physical activities.

### Form 6

This form is designed for physical exercise, sexual activities with or without a vibrator or stimulating system, reading and comfort, and relaxation through the use of a vibrator or a stimulating system to relax the muscles of the back. It is also designed to assist in sexual intercourse. This form is designed to work in relation with the form 6. It is the counter form that fits precisely into the form 6 to allow differing configurations.

The answer to the problem of cushions moving apart from each other while in use, lies simply in creating forms that either do not need more than one cushion to function, and/or creating forms that relate to each other depending on the exercise and fit into one another in such a manner as to limit the possibility of movement between them, and/or creating forms that do not move when used in conjunction with each other due to how they are positioned under the body weight of the user.

Creating forms that do not need other forms to be used is obvious as all of the forms are designed to be useful on their own as well as in conjunction with other forms, whether on the floor next to the form in question, as form 5 was designed to be used, for example or not.

Creating forms that fit well together like a jigsaw, is another useful strategy, as in the relationships between the forms 2 and 3 or the forms 6 and 7. Both of these forms were created in relation to each other to make it so, that although the materials themselves are not adapted to reduce friction, the forms still do not move away from each other during use.

Forms 2 and 3 are designed to relate to each other in just this manner. The radius of the concave middle curve of form 2, (98) is as close to being equal as possible to the radius of the convex side curves on form 3, (100). This method helps to hinder the movement between the two forms enough to hold them in place during use. This is referenced in FIG. 28.

Forms 5 and 6 are also designed to relate to each other in this same manner. The segment (78) on Form 5 is made to be as close to parallel as possible to the segment (92) on the form 6. The segment (80) on form 6 is also meant to be as close to parallel as possible to segment (90) on Form 6 as referenced in FIGS. 22 and 26. The angle between the segments (78) and (80) on Form 5 is less than 160 degrees. This creates a natural positive (102) and negative (104) between the forms 5 and 6 that is sufficient in reducing slippage between the cushions, as can be seen in FIG. 29.

Another useful strategy is to create forms that are held in place by the users themselves. The forms 2 and 3 are a good example of this, as the user of Form 3 straddles the form with their weight bearing down directly upon the form, the users legs are placed on either side, their hands holding it steadily in place.

This would be the same as a child holding his little brother in place during rough play, as many of us know this is a difficult position to wiggle out of. This can be seen in Figure (12)

### Exercise Apparatus

### Form 5 and Form 6

The invention is an interaction of two forms designed to work together as a unit, forming an adjustable weightlifting/exercise system. By using two forms that are designed to interlock into a number of varying configurations, it was found that a great number of the basic exercises could be facilitated.

It should be noted that the preferred embodiment shows a co relation between the Form 6 and the Form 7 where the angle between the segments (78) and (80) on Form 6 is the matching inverse of the angle between the segments (90) and (92) on the Form 7. As referenced in FIG. 29.

Form 5 and Form 6 are primarily but not exclusively for the following uses.
To be placed in the interlocking positions as set out in FIGS. 30, 31, 32, 33, 34, 35, 36 and 37 for the use of physical exercise, weightlifting and other physical activities such as yoga, Pilates and sex.

### Operation

Move the Form 6 into the desired combinations in relation to Form 6 as is referenced in FIGS. 30, 31,32, 33, 34, 35,36 and 37.

### Operation—Alternative Embodiments

### Fluid Collection System

The operation is simple. Detach the removable pocket either fully or partially. Place a pad between said removable pocket and the waterproof cover. Reattach the removable pocket. The invention is now ready for any release of fluids.

When a release of fluid from a user above fills the absorbent pad, detach the removable pocket (FIG. 38) and dispose of the absorbent pad (FIG. 39). Wipe down the waterproof surface with a cloth and possibly a disinfectant and place another absorbent pad onto the waterproof cover. Then replace the removable, water permeable pocket with another clean one. (FIG. 40) The soiled pocket can then be washed for reuse.

The approximate placement of each absorbent pad is set out in FIGS. 41 (106), 42 (108), 43 (110), 44 (112), 45 (114), 46 (116)(118), 47 (120)(122), 48 (124)(126) and 49 (128).

### Stimulation Devices

### Version 1

### Operation

Place the vibrator or stimulator in the desired locale. Place the strap over the device and tighten the strap.

### Version 2

### Operation

The vibrator is pushed through the buttonhole and placed within the underlying pocket.

### Version 3

### Operation

The vibrator is pushed through the buttonhole and placed within the underlying pocket.

### CONCLUSION, RAMIFICATIONS, AND SCOPE

Thus the reader will see that the three dimensional forms within the invention provide comprehensive ergonomic solutions to problems regarding the positioning of the body. And further that they were designed specifically and methodically to help support people in a great number of positions and in a number of different spheres of physical activity.

The invention is lighter, more versatile, and has eliminated the concerns of forms moving away from each other during use. It has taken the concerns of the handicapped as well as the able bodied population into account within its design. The invention helps people use their bodies comfortably, in resting, sex, and in exercise.

### DRAWINGS

### Figures

FIG. 1. Shows a side view of form 1

FIG. 2. Shows a perspective view of form 1

FIG. 3. Shows a possible use for form 1

FIG. 4. Shows a possible use for form 1

FIG. 5. Shows a possible use for form 1

FIG. 6. Shows a possible use for form 1

FIG. 7. Shows a side view of form 2

FIG. 8. Shows a perspective view of form 2

FIG. 9. Shows a possible use for form 2

FIG. 10. Shows a possible use for form 2

FIG. 11. Shows a possible use for form 2

FIG. 12. Shows a possible use for form 2 together with form 3

FIG. 13. Shows a side view of form 3

FIG. 14. Shows a perspective view and form 3

FIG. 15. Shows a possible use for form 3

FIG. 16. Shows a side view of form 4

FIG. 17. Shows a perspective view of form 4

FIG. 18. Shows a possible use for form 4

FIG. 19. Shows a possible use for form 4

FIG. 20. Shows a side view of the diamond extra form

FIG. 21. Shows a perspective view of the diamond extra form

FIG. 22. Shows a side view of the form 5

FIG. 23. Shows a perspective view of the form 5

FIG. 24. Shows a possible use for form 5

FIG. 25. Shows a possible use for form 5

FIG. 26. Shows a side view of the form 6

FIG. 27. Shows a perspective view of the form 6

FIG. 28. Shows the relationship between the contiguous surfaces of form 2 and the form 3

FIG. 29. Shows the relationship between the contiguous surfaces of form 5 and the form 6

FIG. 30. Shows a side view of the possible uses of the form 5 and form 6 used together

FIG. 31. Shows a side view of the possible uses of the form 5

FIG. 32. Shows a side view of the possible uses of the form 5

FIG. 33. Shows a side view of the possible uses of the form 5 and form 6 used together

FIG. 34. Shows a side view of the possible uses of the form 5

FIG. 35. Shows a side view of the possible uses of the form 5 and form 6 used together

FIG. 36. Shows a side view of the possible uses of the form 6

FIG. 37. Shows a side view of the possible uses of the form 5 and form 6 used together

FIG. 38. Shows a perspective view of the support device, removable pocket, absorbent pad and waterproof cover, with removable pocket partially open

FIG. 39. Shows a perspective view of the support device, removable pocket, absorbent pad and waterproof cover, with removable pocket partially open and the absorbent pad being placed under an open removable pocket

FIG. 40. Shows a perspective view of the support device, removable cover, and waterproof cover, with removable pocket fully closed

FIG. 41. Shows the location for the placement of the removable pocket and absorbent pad upon the form 1

FIG. 42. Shows the location for the placement of the removable pocket and absorbent pad upon the extra diamond form

FIG. 43. Shows the location for the placement of the removable pocket and absorbent pad upon the form 2

FIG. 44. Shows the location for the placement of the removable pocket and absorbent pad upon the form 3

FIG. 45. Shows the location for the placement of the removable pocket and absorbent pad upon the form 4

FIG. 46. Shows the location for the placement of the removable pocket and absorbent pad upon the form 5 additional embodiment

FIG. 47. Shows the location for the placement of the removable pocket and absorbent pad upon the form 5 preferred embodiment

FIG. 48. Shows the location for the placement of the removable pocket and absorbent pad upon the form 6

FIG. 49. Shows the location for the placement of the removable pocket and absorbent pad upon the form 6

FIG. 50. Shows the location for the placement of the stimulating device upon the form 1

FIG. 51. Shows the location for the placement of the stimulating device upon the form 2

FIG. 52. Shows the location for the placement of the stimulating device upon the form 3

FIG. 53. Shows the location for the placement of the stimulating device upon the form 4

FIG. 54. Shows the location for the placement of the stimulating device upon the form 5 additional embodiment

FIG. 55. Shows the location for the placement of the stimulating device upon the form 5 preferred embodiment

FIG. 56. Shows the location, operation and placement of the stimulating device upon the form 3 using a strap method

FIG. 57. Shows the location, operation and placement of the stimulating device upon the form 3 using a strap method

FIG. 58. Shows the strap and the nonslip material

FIG. 59. Shows the location, operation and placement of the stimulating device upon the form 1 using a pocket method

FIG. 60. Shows the location, operation and placement of the stimulating device upon the form 1 using a pocket method

1. What is claimed is a bodily support system comprised of one or more specific three dimensional forms made of foam, the dimensions and characteristics of which are as follows,
A) form 1, as described by using points of reference upon the surface of said three dimensional form, corresponding to points within an x, y axis where x is on the horizon and y is on the vertical and the depth is between 11 and 30 inches, and whereas, each point can vary in its location on the X and Y axis by 1 inch from its described point, in any direction on the X and Y axis,
Point 2 is at the origin,
Point 4 is at X=0″, Y=5 and 7/16″
Point 6 is at X=3″ and 5/16″ and Y=7 and ¼″
Point 8 is at X=5″ and 9/16″ and Y=4 and ¾″
Point 10 is at X=7″ and ¾″ and Y=3 and 7/16″
Point 12 is at X=10″ and Y=2 and 9/16th″
Point 14 is at X=12″ and ¼″ and Y=2″
Point 16 is at X=13″ and ½″ and Y=1 and 1/16″
B) form 2, as described by using points of reference upon the surface of said three dimensional form, corresponding to points within an x, y axis where x is on the horizon and y is on the vertical and the depth is between 11 and 30 inches, and whereas, each point can vary in its location on the X and Y axis by 1 inch from its described point, in any direction on the X and Y axis,
Point 20 is at the origin,
Point 22 is at X=0, Y=4 and ⅜″
Point 24 is at X=6 and ⅝″ and Y=7 and ⅝″
Point 26 is at X=10 and 1/16″ and Y=7 and 1/16″
Point 28 is at X=13 and 7/16″ and Y=5 and 13/16″
Point 30 is at X=16 and ¾″ and Y=4 and ¾″
Point 32 is at X=20″ and Y=4 and ⅜″
Point 34 is at X=23 and ¼″ and Y=4 and ¾″
Point 36 is at X=26 and 9/16″ and Y=5 and 13/16″
Point 38 is at X=29 and 15/16″ and Y=7 and 1/16″
Point 40 is at X=33 and ⅜″ and Y=7 and ⅝″
Point 42 is at X=40″ and Y=4 and ⅜″
C) form 3 is a three sided triangular form which possesses side segments, with a radius of between 14″ and a straight line as well as a height which is between 9″ and 12″ from the middle point on one of the side segment to intersecting point of the other two segments, with a depth of between 12″ and 30″,
D) form 4 is described as a segment of between 9″ and 12″ in length at between 22 to 30 degrees in relation to another segment of between 9″ and 12″ in length that are joined with a radius of between ¾″ and 2″he segments are also connected at the opposite ends with a radius of between 3″ and 5″, with a width of the form is between 19″ and 30″,
E) form 5 is described as a segment between 35″ to 55″ in length which intersects segment (76) which has a length of between 5″ and 12″ with an angle between 60 to 90 degrees rising counter clockwise to the horizon. A segment (78) of between 9″ and 15″ in length intersecting segment (76) at an angle of between 15 and 45 degrees rising clockwise in relation to the horizon. Segment (78) also intersects segment (80) which has a length of between 20″ and 30″ at an angle of between 15 and 35 degrees rising counterclockwise from the horizon. Segment (80) intersects segment (82). Segment (82) has a length of between 0″ and 6″ and can be between 0 and 90 degrees rising clockwise from the horizon. Segment (82) intersects segment (84). Segment (84) has a length of between 4″ and 20″ and in between 60 and 90 degrees rising from the horizon. Segment (84) intersects segment (74), as referenced in FIG. 22,
F) form 6 is described as a Segment (88) that is between 24″ and 38″ in length and intersects segment (90) with an angle of between 15 and 35 degrees. Segment (90) is between 15″ and 26″ in length. Segment (92) is between 9″ and 15″ in length and intersects segment (90). The angle between (90) and (92) is as close as possible to the angle chosen between segments (78) and (80), somewhere in the range between 110 to 160 degrees. Segment (94) is between 0″ and 7″ in length and intersects (92) at an angle between 60 and 110 degrees. Segment (94) intersects segment (88) with an overall width of between 12′ and 36″, as referenced in FIG. 26.

2. The three dimensional forms of claim 1 may be made entirely or in part of a poly urethane foam, latex rubber, closed cell foam or a polyethylene foam.

3. The three dimensional forms of claim 1 may be made entirely or in part of a body supporting material.

4. The three dimensional forms of claim 1 may be enclosed in a cover made entirely or in part of a cotton, nylon or polyester material.

5. The three dimensional forms of claim 1 may have a stimulating or relaxation device attached them by means of a pocket that holds said device in place.

6. The three dimensional forms of claim 1 may have a stimulating or relaxation device attached them by means of a strap that holds said device in place.

7. What is claimed is a system for the collection of bodily fluids comprised of,
A.) a bodily support device made of foam.
B.) a water proof cover encasing the bodily support device,
C.) a removable cover made of a water permeable material.
D.) a fastening device attached to both said removable cover and said water proof cover in such a way as to create a space under said removable cover in which to place a water absorbent pad.

8. The bodily support device of claim 7 may be made entirely or in part of a poly urethane foam, latex rubber, closed cell foam or a polyethylene foam.

9. The three dimensional forms of claim 7 may be made entirely or in part of a body supporting material.

10. The waterproof material of claim 7 may be made entirely or in part out of a cotton, nylon or polyester material.

11. The water permeable material of claim 7 may be made entirely or in part out of a cotton, nylon or polyester material.

12. The fastening device of claim 7 may be a zipper, hook and loop, group of buttons or any other suitable fastening device.

13. What is claimed is an exercise apparatus comprised of two interlocking, three dimensional forms made of a supporting foam, that can be rearranged into various contiguous, body supporting formations, the dimensions and characteristics of which are as follows,
A.) form 5 is described as a segment between 35″ to 55″ in length which intersects segment (76) which has a length of between 5″ and 12″ with an angle between 60 to 90 degrees rising counter clockwise to the horizon. A segment (78) of between 9″ and 15″ in length intersecting segment (76) at an angle of between 15 and 45 degrees rising clockwise in relation to the horizon. Segment (78) also intersects segment (80) which has a length of between 20″ and 30″ at an angle of between 15 and 35 degrees rising counterclockwise from the horizon. Segment (80) intersects segment (82). Segment (82) has a length of between 0″ and 6″ and can be between 0 and 90 degrees rising clockwise from the horizon. Segment (82) intersects segment (84). Segment (84) has a length of between 4″ and 20″ and in between 60 and 90 degrees rising from the horizon. Segment (84) intersects segment (74), as referenced in FIG. 22
B.) form 6 is described as a Segment (88) that is between 24″ and 38″ in length and intersects segment (90) with an angle of between 15 and 35 degrees. Segment (90) is between 15″ and 26″ in length. Segment (92) is between 9″ and 15″ in length and intersects segment (90). The angle between (90) and (92) is as close as possible to the angle chosen between segments (78) and (80), somewhere in the range between 110 to 160 degrees. Segment (94) is between 0″ and 7″ in length and intersects (92) at an angle between 60 and 110 degrees. Segment (94) intersects segment (88) with an overall width of between 12′ and 36″, as referenced in FIG. 26.

14. The three dimensional forms of claim 13 may be made entirely or in part of a poly urethane foam, latex rubber, closed cell foam or a polyethylene foam.

15. The three dimensional forms of claim 13 may be made entirely or in part of a body supporting material.

16. The three dimensional forms of claim 13 may be enclosed in a cover made entirely or in part of a cotton, nylon or polyester material.

17. The three dimensional forms of claim 13 may have a stimulating or relaxation device attached them by means of a pocket that holds said device in place.

18. The three dimensional forms of claim 13 may have a stimulating or relaxation device attached them by means of a strap that holds said device in place.
